(19) United States
(12) Patent Application Publication
(10) Pub. No.: US 2010/0227008 A1
(43) Pub. Date: Sep. 9, 2010

(54) SALICYLIC ACID ACNE SPRAY FORMULATIONS AND METHODS FOR TREATING ACNE WITH SAME

(75) Inventor: Amy E. BAKER, Oakland, CA (US)

Correspondence Address: MORRISON & FOERSTER LLP, 755 PAGE MILL RD, PALO ALTO, CA, 94304-1018, US

(73) Assignee: Nature's Cure, Oakland, CA (US)

(21) Appl. No.: 12/702,211

(22) Filed: Feb. 8, 2010

Related U.S. Application Data
(63) Continuation of application No. 11/648,369, filed on Dec. 28, 2006, now abandoned.
Application No. 11/648,369 is a continuation of application No. 09/557,187, filed on Apr. 21, 2000, now abandoned.

(51) Int. Cl. A61K 36/54; A61P 17/10

(52) U.S. Cl. 424/739

(57) ABSTRACT

A fine mist spray of a salicylic acid solution is provided for treating body acne. The pH of the solution may be adjusted to above about 4.5 to reduce the incidence of nasal irritation and coughing due to incidental inhalation of the spray.

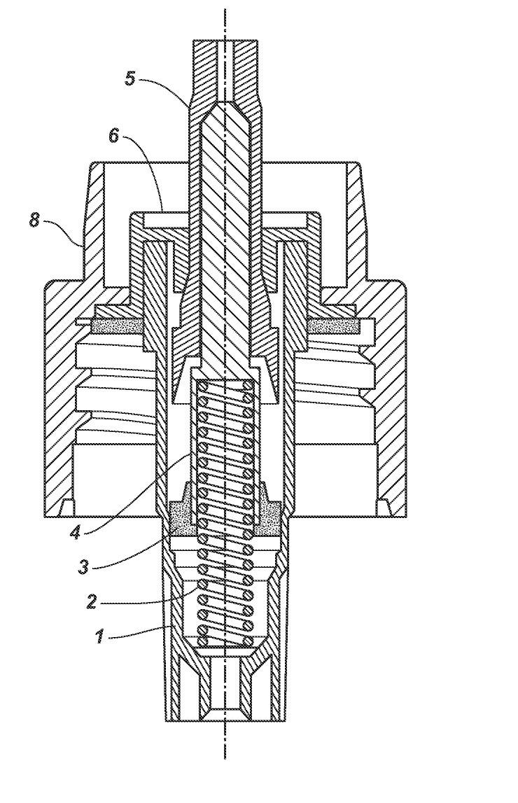

### CROSS-REFERENCE TO RELATED APPLICATION

The present application is a continuation of U.S. patent application Ser. No. 11/648,369, filed on Dec. 28, 2006 and which claims priority from U.S. patent application Ser. No. 09/557,187, filed on Apr. 21, 2000, each of which is incorporated herein by reference in their entirety.

### FIELD OF THE INVENTION

The present invention relates to the field of pharmaceutical arts. More specifically, the invention relates to spray formulations comprising salicylic acid to be administered topically to skin for the treatment of acne or acneform conditions.

### BACKGROUND ART

The use of salicylic acid in the treatment of common or teen acne is known. For example, U.S. Pat. No. 4,665,063 describes the use of topically applied aspirin (acetyl salicylic acid) for treating common acne; and U.S. Pat. No. 4,891,227 describes the use of pads for applying anti-acne products containing salicylic acid for oily skin. These patents describe state-of-art compositions which emphasize aggressive chemical and physical treatment suitable for teen acne, without addressing the suitability for adult acne and/or the need for mildness.

U.S. Pat. No. 4,800,197 describes a combination of salicylic acid and an anionic taurate surfactant, specifically sodium methyl cocoyl taurate or sodium methyl oleoyl taurate. U.S. Pat. No. 5,296,476 describes the specific use of salicylic acid in combination with calcium citrate. Again, these treatment modalities are designed for aggressive, physical cleansing, which assumes that the individual indicators are normal, young and oily skin.

Currently available forms of salicylic acid tend to aggravate the relatively dry adult acne, and they are particularly unsuitable for those with sensitive skin conditions such as irritant folliculitis. Known salicylic acid preparations are also poorly tolerated in patients suffering from acne complexed with rosacea.

U.S. Pat. No. 5,569,651 teaches the use of a salicylic acid cream and lotions whose pH is adjusted to from about 3.8 to 4.5 using ammonium hydroxide. U.S. Pat. No. 5,871,764 discloses a salicylic acid powder formulation having a pH of from about 3 to about 4. U.S. Pat. No. 5,612,324 discloses salicylic acid solutions, gels and pads having a pH of from about 2 to about 6.5.

Additional examples of salicylic acid compositions wherein pH of the composition was controlled may be found in the following U.S. Pat. No. 5,756,119 (pH of less than 5); U.S. Pat. No. 5,549,888 (pH of 2-5.4); U.S. Pat. No. 4,294,852 (pH of 2-6.5); U.S. Pat. No. 5,702,688 (pH less than or equal to 4.2); 4,800,197 (pH of 2-3.5); and U.S. Pat. No. 5,958,436 (pH of 1-6).

While several salicylic acid formulations have been disclosed in the prior art for treating acne, applicants are not aware of the disclosure of a specific spray formulation of salicylic acid. In this regard U.S. Pat. No. 5,612,324 suggests formulating salicylic acid and dexpanthenol in an emulsion having a consistency of a thin lotion which can be suitable for spray or aerosol delivery. No specific formula is, however, suggested. Similarly, U.S. Pat. No. 5,958,436 describe skin exfoliant formulations of salicylic acid and calcium ions and suggest the formulations may be administered by spraying (including mist, aerosol or foam spraying). Accordingly, applicants believe that the art has not fully appreciated the problems associated with developing a salicylic acid formulation suitable for administration as a fine mist spray.

### DISCLOSURE OF THE INVENTION

The present invention provides a fine mist acne spray comprising a solution of salicylic acid wherein the salicylic acid constitutes from about 0.01% to about 20% by weight of the solution. The pH of the formulation will preferably be selected to reduce the likelihood that the spray will cause irritation of the nasal passage or coughing.

An article of manufacture for treating acne is also provided which comprises a salicylic acid formulation as described above contained in a fine mist spray dispenser.

Methods are also provided for treating acne comprising administering a fine mist spray of the above described formulation to a human skin surface afflicted with acne or an acneform condition.

### BRIEF DESCRIPTION OF THE DRAWING

The drawing is a sectional view of the pump element of a fine mist spray pump dispenser.

### MODES FOR CARRYING OUT THE INVENTION

### A. Definitions

As used in the specification and claims, the singular form a, an and the include plural references unless the context clearly dictates otherwise. For example, the term a carrier may refer to one or more carriers for use in the presently disclosed formulations and methods.

As used herein the term “acne” refers to any and all forms of acne as well as acneform conditions such as, without limitation, folliculitis keratosis pilaris. Acne is a broad clinical syndrome most frequently occurring at puberty in both men and women. It may last through life, and consists of lesions most typically on the face and trunk, and consists of papules, pustules, comedones (open and closed), cysts, and microcysts.

The term pharmaceutically acceptable means that the ingredient that is being qualified is compatible with the other ingredients of the formulation and not injurious to the patient. Several pharmaceutically acceptable ingredients are known in the art and official publications such as THE UNITED STATES PHARMACOEPIA describe the analytical criteria to assess the pharmaceutical acceptability of numerous ingredients of interest.

The term pharmaceutical carrier or simply carrier as used herein refers to a composition that contains and or delivers a pharmacologically active agent and is generally considered to be otherwise pharmacologically inactive. However, the carriers of this invention may have some therapeutic effect when applied to a site such as skin, by providing, for example, protection to the site of application from conditions such as injury, further injury, or exposure to elements.

The terms aerosol and fine mist spray are used interchangeably throughout this disclosure and refer to a mixture of liquid particles in a gas wherein the size of the liquid particles are in the range from about 10-150 micrometers.

A propellant is a substance that is a gas under atmospheric conditions but a liquid when under pressure that is used to generate a fine mist spray. Pharmaceutically acceptable propellants include, but not limited to, the following: dimethyl ether; diethyl ether; fluorocarbons such as propellant 152a (1,1-difluoroethane, also known as Dymel®), a hydrocarbon, a liquefied gas such as nitrogen or carbon dioxide or a mixture thereof.

The propellant preferably comprises from about 5% to about 70% by weight of the final formulation, and more preferably from about 30% to about 60% by weight of the final formulation. When actuated, the pressure difference between the inside of the container and outside causes the rapid expansion of the propellant molecules and the ejection of the contents as a spray.

A pump spray is a formulation that does not contain a propellant and is ejected from a closed container by means of mechanical force (i.e., pushing down a piston with one's finger or by compression of the container, such as by a compressive force applied to the container wall or an elastic force exerted by the wall itself (e.g. by an elastic bladder)). For examples and applications of pump sprays, see S. Borum, et al., Comparison Between the Effect of Ipratropiurn Bromide as a Pressurized Aerosol and as an Aqueous Pump Spray on Methacholine-induced Rhinorrhea, Rhinology 34(4): 198-200 (1996); M. Daublander, et al., Clinical Investigation of Potency and Onset of Different Lidocaine Sprays for Topical Anesthesia in Dentistry, Anesth. Pain Control Dent., 1(1): 25-28 (1992); and A. S. Harris, et al., Effect of Viscosity on Particle Size, Deposition, and Clearance of Nasal Delivery Systems Containing Desmopressin, J Pharm. Sci., 77(5): 405-408 (1988).

A solvent system is a mixture of at least one volatile solvent and water.

The term volatile refers to the characteristic of evaporating within a short time at ambient temperatures or at the temperature of a live human body. Thus a solvent is volatile if it evaporates at temperatures below 40° C. and preferably within about a few seconds to about two minutes.

A pH adjuster refers to an agent to adjust the pH of the present formulations to a desired level or range. The pH adjuster can be a buffer, a base or an acid, or a combination thereof. The pH adjuster preferably comprises between about 0.01% and about 20% by weight of the final formulation, and more preferably between about 0.01% and 10% by weight of the final formulation. Some examples of bases include, but not limited to, sodium hydroxide, potassium hydroxide, and low molecular weight amines, organic substituted amines such as substituted alkyl amines, such as triethanolamine. Some examples of acids include inorganic acids such as hydrochloric acid, and organic acids such as acetic acid, lactic acid, citric acid, tartaric acid, etc. Buffers include phosphate buffers, citrate buffers, sulfate buffers etc, which are well-known in the art.

An effective amount is an amount sufficient to effect beneficial or desired results with respect to treating acne and/or acneform conditions. An effective amount can be administered in one or more administrations.

Concentrations, amounts, etc., of various components of this invention and its pH values are often presented in a range format throughout this application. The description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as 6% to 12% should be considered to have specifically disclosed subranges such as 6% to 7%, 7% to 8%, 7% to 9%, 6% to 9%, 9% to 12%, 9% to 11% etc., as well as individual numbers within that range, for example, 8%, 10%, 11% etc. This applies regardless of the breadth of the range and in all contexts throughout this application. Unless otherwise indicated, percentages are intended to be by weight.

### B. The Formulation

The salicylic acid is present in the solution of from about 0.01% to about 20% by weight, or, preferably, from about 0.1% to about 7% by weight, or, most preferably, from about 0.5% to about 2% by weight. The salicylic acid will typically be dissolved in a pharmaceutically acceptable carrier. The carrier will constitute from about 0.1% to about 99.8% by weight of the formulation preferably from about 80% to about 99% and most preferably from about 85% to about 95%. An especially preferred carrier for pump sprays is a hydroalcoholic solvent system comprising from about 1% to about 99% of a lower alcohol such as denatured ethanol, and from about 1% to about 99% of water. More preferred is a carrier comprising from about 5% to about 60% of denatured ethanol, and from about 40% to about 95% of water. Especially preferred is a carrier comprising from about 20% to about 50% of denatured ethanol, and from about 50% to about 80% of water.

In the case of spray formulations that use a propellant, the aerosol propellants may not be that freely miscible with water. To improve its miscibility with water, a co-solvent such as ethanol, 2-propanol, dimethyl ether or acetone may be used in order to produce a clear solution or a partial solution-suspension system. See, REMINGTON, supra, Chapter 95, page 1682, left column.

The formulation may optionally contain additional anti-acne ingredients to salicylic acid. Examples of such ingredients are: other keratolytic agents such as benzoyl peroxide and α-hydroxyacids such as retinoic acid or derivatives thereof; other anti-acne retinoids such as adapalene, tazaretene; antimicrobials such as penicillins, cephalosporins, other beta-lactams, aminoglycosides, tetracyclines, erythromycin, clindomycin and other antifungal agents; antiseptics such as triclosan, phenoxyisopropanol, resorcinol, chlorhexidine, povidone, and iodine; anti-irritants such as a-bisabolol, farnesol, chamomile extract and glycyrrhetinic acid; and other common anti-acne compositions such as urea, allantoin, glycolic acid, azelaic acid and hydroxyquinolines.

The formulation may further contain fragrances, solubility agents, vitamins, natural extracts, and other ingredients commonly found in topical formulations as is known in the art.

pH:

Since the spray will typically be used on non-facial body skin, e.g., back, arms, neck, and chest, and consumers frequently want to treat the afflicted skin area and then clothe themselves quickly, the invention spray is a fine mist spray that can be focused onto a small target skin area and dries quickly. One problem associated with fine mist sprays is that some of the spray particles may incidentally enter the nasal passages and throat and cause irritation or coughing. According to one aspect of the invention, the likelihood of inducing such irritation and coughing in the user population can be significantly lessened by increasing the pH of the spray. In this regard, the prior art appears to discourage one from raising the pH of a topical salicylic acid formulations beyond the pH range of the upper layers of the skin. While the upper layers of skin (epidermis and stratum corneum) have a pH of 4.2 to 5.6, salicylic acid has a pH of about 2.4 and a pKa of about 2.9. This pH differential can cause breakdown of the stratum corneum, resulting in severe irritation and skin damage over multiple applications. See, for example, U.S. Pat. No. 5,702,688.

Two factors, perhaps interrelated, appear to discourage the pursuit of a salicylic acid formulation with a pH higher than these levels. First, it has been suggested that a pH around 7, or greater than 7 would diminish the anti-acne efficacy of acidic active agent formulations in general (see U.S. Pat. No. 4,507,319, Column 4, lines 26-38) and salicylic acid formulations in particular (see the U.S. Pat. No. 5,702,688, col. 2, lines 39-55). Thus, most of the specific examples disclosed in the patent literature show a pH of less than 6.0. Second, as the pH of the formulation increases, the amount of salicylic acid (with a pKa of about 2.9) that is in the ionized form increases, thereby decreasing the amount of salicylic acid that actually crosses the skin. It is generally thought that salicylic acid crosses the skin at a greater percentage as unionized species. Thus, an acidic formulation range is art-preferred for salicylic acid compositions in order to suppress ionization and enhance its penetration into the stratum corneum.

However, it has been discovered in the instant invention that a balance can be struck between the pH of the formulation and the particle size of the spray without sacrificing the anti-acne efficacy of the formulations.

In order to lessen the likelihood of causing nasal/throat irritation and coughing, it is preferable that the spray have a pH above about 4.5, usually about 4.5 to about 7.5. More preferable, the pH may range from about 5 to about 7.2. Even more preferably, the pH is substantially neutral, i.e., from about 6.9 to about 7.2. A variety of acids, bases, and buffers can be used to adjust and/or maintain the pH of the spray. Triethanolamine is a preferred agent to adjust the pH of the present salicylic acid sprays. However, other nonlimiting examples of agents useful include sodium carbonate, sodium hydroxide, hydrochloric acid, phosphoric acid, sodium hydrogen phosphate, sodium dihydrogen phosphate, citric acid, and the like.

Dispensers

The fine mist sprays of this invention may be dispensed from propellant-based dispensers or from pump spray dispensers. These dispensers comprise a container that contains the spray formulation, a fine mist nozzle assembly affixed to the top of the container through which the formulation is dispensed, and a pressure generator that exerts pressure on the liquid formulation to cause it to be expelled from the nozzle. The pressure generator may be a propellant contained in the container that exerts pressure on the liquid formulation, a pump assembly, or an elastomer bladder in which the liquid formulation is contained. Preferably, the pressure generator is a pump assembly that is adapted to screw onto the neck of a plastic bottle that is adapted to hold the salicylic acid solution. Such pumps may be purchased commercially from Emsar, Inc., Pfeiffer, or Calmar. The drawing shows the details of such a pump assembly.

Referring to the drawing, the pump assembly comprises a housing 1, a housing cap 6 sealingly affixed about one end of the housing which in turn is affixed to a screwcap 8 that is structured to be screwed onto the neck of a bottle (not shown) which holds the salicylic acid spray formulation. The housing provides a reservoir for holding portions of the formulation and contains a hollow piston 5 that may be manually depressed to exert pressure on the liquid contained in the housing thereby forcing it up through the bore in the piston and a nozzle (not shown) affixed to the top of the piston. The lower end of the piston carries a stem 4. A sliding seal 3 is positioned between the inner wall of the housing and the stem. A spring resides in the lower portion of the housing between the housing and the lower end of the piston for the purpose of forcing the piston upward after manual force has been removed from the top of the piston. A dip tube (not shown) extends from the bottom of the housing down into the liquid for transporting liquid from the container into the housing reservoir.

The nozzle preferably provides a full cone spray pattern wherein the area encompassed by the pattern is completely filled with spray drops. The outline of the area is preferably circular but may be other shapes.

The dispenser is typically sized to contain from 50 to 500 ml of the liquid spray formulation. The pump assembly will typically be designed to expel between 10 to 1500 μL of liquid per actuation, more usually 50 to 500 μL and even more usually from 50 to 150 μL.

Because the spray is intended primarily for application to non-facial portions of the body, the pump assembly is preferably one that can be operated right-side up, upside down or any position therebetween. Such spray dispensers are commonly referred to as 360 degree spray dispensers.

### F. Examples

The following Examples further illustrate the invention and are not intended to limit the invention in any manner.

### Example 1

### Preparation and Analysis of the Formulations

a) Preparation

A formulation comprising salicylic acid was formulated. The ingredients of these formulations are shown in Table 1 below.

TABLE 1

| Ingredients | Range (% by Volume) | Source |
|---|---|---|
| Salicylic acid | 2% | Spectrum |
| Allantoin | A | Ingredients Int. |
| Aloe Vera | A | New Age Botanical |
| Burdock Extract | A | Vege Tech |
| Calendula Extract | A | Vege Tech |
| Capryloylglycine/ | A | Seppic Int. |
| Methylglycine | | |
| Cinnamon Extract | | |
| Chamomile Extract | A | Vege Tech |
| Coneflower Extract | A | Active Organics |
| Deionized Water | C | |
| Diazolidinyl Urea/ | B | Sutton Lab |
| Methylparaben/ | | |
| Propylene Glycol/ | | |
| Propylparaben | | |
| Fragrance | A | |
| SD Alcohol 40* | C | Remet |
| Sodium Citrate | A | VWR Chemical |
| Tocopheryl Acetate | A | Hoffmann La Roche |
| (vitamin E) | | |
| Triethanolamine | B | VWR Chemical |
| Willow Bark Extract | A | Brook's Industry |
| Witch Hazel | A | Vege Tech |

A = <1% to 5%
B = 1% to 10%
C = >10%
*Ethyl alcohol denatured with t-butyl alcohol and any combination of one or more of: brucine, brucine sulfate or quassin.

The alcohol, salicylic acid and tocopheryl acetate were premixed together until all solids were dissolved. The other ingredients (except for fragrance) were added one at a time to the water with continuous stirring. The alcohol solution was then added to the water solution, mixed well and filtered. The fragrance was then added to the formulation.

The formulation was then placed into a plastic bottle. The bottle was fitted with an Emsar 37 MS 24/240 2-way pump which can deliver approximately 120 μL+/−10 μL per actuation. This 2-way pump comprised of an Emsar 2171-060 dip tube with a 4¾″ A06 actuator with a 2762-1609 insert. Other dispensers with 2762-1310, 2762-1510, 2762-2015, and 2762-2040 inserts were also prepared.

b) Analysis

The formulations were analyzed for the characteristics of volume released per actuation, spray pattern and particle size and for leakage.

i) Particle Size:

Particle size was measured using a Malvern particle sizer. The data are shown in Table 2 below.

TABLE 2
Median Particle Size (Micrometers) and Insert Type

| 2762-1310 | 2762-1510 | 2762-1609 | 2762-2015 | 2762-2040 |
|---|---|---|---|---|
| 63 | 62 | 66 | 70 | 81 |

Numbers shown are the averages of measurements from three samples.

As shown in Table 2, median particle size was fairly consistent for each type of insert, and varied only by a maximum of about 3 micrometers.

ii) Accelerated Stability Testing for Consistency in Delivery Volume:

Accelerated stability testing is a standard method in the art of pharmaceutical sciences. See generally, Remington supra, Chapters 18 and 38.

Accelerated stability testing was also performed by storing the dispensers at 110° F. and measuring the volume released per actuation. These data indicate that, after one week, all dispensers continued to work satisfactorily with a less than 4-5% variation. The data are presented in Table 3.

TABLE 3
Volume Released (μL) per Actuation from Accelerated
Stability Testing

| Volume (μL) Released Per Actuation | Average | High | Low |
|---|---|---|---|
| Initial | 124 | 130 | 121 |
| 1 Week | 121 | 127 | 117 |

iv) Leakage Testing:

After the above-described accelerated temperature testing, all dispensers were inverted and the pumps stoked eight times. No unacceptable leakage was noted.

### Example 2

### Effect of pH on Irritation/Coughing

An experiment was performed to study the effect of pH on coughing by a subject using the fine mist spray formulations of the invention. Several formulations of the present invention with varying pH values were prepared by the methods described above (pH: 7.0, 6.0, 5.09, 4.06, 3.0 and 2.6). The dispensers were fitted with an Emsar 2-way pump 2171-060 having a dip tube of 4¾″ and an A06 actuator with a 2762-1609 insert that can provide a substantially uniform spray pattern with a particle size range of about 60-70 micrometers. These dispensers can deliver approximately 120 μL+/−10 μL volume per actuation. Seven normal healthy volunteer subjects were selected at random and were asked to spray one formulation at a time, starting with the highest pH sample (pH 7.0 in this case) and then walk through the spray mist breathing normally. The experiment was repeated three to five times.

In these tests the incidence of irritation/coughing began at pHs in the range of 5-6. These tests, therefore indicate that pHs above about 4.5 are desirable to reduce the incidence of nasal irritation and coughing.

Modifications of the above-described modes for carrying out the invention that are obvious to persons of skill in the pharmaceutical, cosmaceutical, or related arts are intended to be within the scope of the following claims.

What is claimed is:

1. A method of treating non-facial acne on a person in need thereof comprising:
delivering a fine mist acne-treatment formulation to a non-facial body region, wherein the acne-treatment formulation comprises:
0.01% to 20% by volume of Salicylic acid;
1% to 40% by volume of natural extracts;
and a solvent system comprising water and a volatile solvent, wherein the formulation has a pH in the range from about 5 to about 7.2;
and wherein the delivery of the acne-treatment formulation to the non-facial body region results in the person experiencing less nasal irritation and less coughing than would be experienced by applying a fine mist acne-treatment formulation having a pH lower than about 5 to the same non-facial body region.

2. The method of claim 1, wherein the formulation comprises about 0.1% to about 7% Salicylic acid.

3. The method of claim 1, wherein the formulation comprises a fragrance.

4. The method of claim 1, wherein the natural extracts comprise: Burdock Extract, Willow Bark Extract, Witch Hazel, Coneflower Extract, Chamomile Extract, Cinnamon Extract, Calendula Extract, and Aloe Vera.

5. The method of claim 1, wherein the Salicylic acid is approximately 2% by volume.

6. The method of claim 1, wherein the dispenser dispenses about 10 to 1500 microliters of solution per actuation.

7. The method of claim 1, wherein the fine mist pump dispenser is a 360 degree fine mist spray pump spray dispenser.

8. The method of claim 1, wherein the dispenser dispenses about 50 to 150 microliters of solution per actuation.

9. The method of claim 1 wherein delivering the fine mist of the acne-treatment formulation comprises delivering the fine mist using a non-propellant fine mist pump spray bottle.

10. The method of claim 9, wherein the acne-treatment formulation comprises about 0.05% to about 2% Salicylic acid.

11. The method of claim 9, wherein the acne-treatment formulation comprises a 2% Salicylic acid formulation.

12. A formulation for the treatment of acne, the formulation comprising:
a solution of Salicylic acid, wherein the salicylic acid constitutes from about 0.01% to 20% of Salicylic acid by weight of the solution, and has a pH of greater than 7;
1% to 40% by volume of natural extracts; and
a fragrance.

13. The formulation of claim 12, wherein formulation comprises about 2% Salicylic acid.

14. The formulation of claim 12, wherein the natural extracts consist of: Burdock Extract, Willow Bark Extract, Witch Hazel, Coneflower Extract, Chamomile Extract, Cinnamon Extract, Calendula Extract, and Aloe Vera.

15. A formulation for the treatment of acne, the formulation comprising:
0.01% to 20% of Salicylic acid
1% to 5% Allantoin
1% to 5% Aloe Vera
1% to 5% Burdock Extract
1% to 5% Calendula Extract
1% to 5% Capryloylglycine/Methylglycine
1% to 5% Cinnamon Extract
1% to 5% Chamomile Extract
1% to 5% Coneflower Extract
1% to 10% Diazolidinyl Urea/Methylparablen/Propylene Glycol/Propylparaben
1% to 5% fragrance
1% to 5% Sodium Citrate
1% to 5% Vitamin E
1% to 10% Triethanolamine
1% to 5% Willow Bark Extract
1% to 5% Witch Hazel
>10% Water
>10% Alcohol
wherein the pH of the formulation is greater than 5 and wherein the percentages refer to percent by volume.

16. The formulation of claim 15, wherein the pH of the formulation is greater than 7.
